(19) United States
(12) Patent Application Publication
(10) Pub. No.: US 2006/0205026 A1
(43) Pub. Date: Sep. 14, 2006

(54) Parallel inducible cell-based kinase screen

(75) Inventors: Zhi Hong, Aliso Viejo, CA (US); William Ricketts, Irvine, CA (US); Paul Diaz, Riverside, CA (US)

Correspondence Address: BROWN, RAYSMAN, MILLSTEIN, FELDER & STEINER LLP, 900 THIRD AVENUE, NEW YORK, NY, 10022, US

(73) Assignee: Valeant Research & Development, Costa Mesa, CA (US)

(21) Appl. No.: 10/540,277

(22) PCT Filed: Dec. 19, 2002
(86) PCT No.: PCT/US02/40757

§ 371 Date: Mar. 27, 2006

(51) Int. Cl. C12Q 1/48; G06F 19/00

(52) U.S. Cl. 435/15; 702/19

(57) ABSTRACT

A parallel inducible cell-based kinase screen (PICKS) includes a plurality of cells that express a kinase gene under the control of an inducer and a reporter gene, wherein the cells are derived from a single cell line, and wherein the reporter gene generate a signal in response to catalytic activity or inhibition of the expressed kinases. Particularly preferred systems include cells expressing kinases from a single kinase signaling pathway, kinases from a kinase family, and/or kinases from various different kinase signaling pathways. Consequently, contemplated systems provide a platform for screening for novel kinase inhibitors, inhibition specificity of particular kinase inhibitors, and for analysis of inter-pathway and/or intra-pathway inhibition of a kinase inhibitor.

### FIELD OF THE INVENTION

The field of the invention is cell-based high-throughput screening as it relates to kinase inhibition.

### BACKGROUND OF THE INVENTION

Protein phosphorylation is involved in numerous regulatory events within a cell. For example, specific phosphorylation of various proteins (mediated through phosphorylation by kinases, or dephosphorylation by phosphatases) often provides a mechanism through which cell surface signaling pathways transmit and integrate information into the nucleus. Protein phosphorylation commonly occurs on the hydroxy group of an amino acid (e.g., tyrosine, serine, or threonine) within a polypeptide, and changes in the phosphorylation state of these polypeptides regulate many aspects of cellular metabolism, growth, and/or differentiation.

Thus, it is not surprising that defects in protein kinases and phosphatases or their regulation are often associated with various diseases. For example, over-expression of certain cellular tyrosine kinases, and particularly the EGF (epidermal growth factor) receptor or the related receptor, HER2 result in cellular transformation. Mutations in a tyrosine kinase that lead to a constitutively active form have been reported in many neoplastic diseases. For example, mutation of the Abl tyrosine kinase is the primary cause of CML. In this case, a chromosomal translocation occurs that produces a fusion protein of Abl and another protein, BCR, resulting in dysregulation of Abl function. In other, non-neoplastic conditions, abnormalities in various protein tyrosine kinases have been implicated in inflammatory signals (Nature Medicine (1996) 2: 561-56). In still other examples, defective Serine/threonine kinase genes have been implicated in several diseases, including myotonic dystrophy and Alzheimer's disease (Sanpei et al. (1995) Biochem. Biophys. Res. Commun. 212: 341-6; Sperber et al (1995) Neurosci. Lett. 197: 149-153; Grammas et al (1995) Neurobiology of Aging 16: 563-569; Govoni et al. (1996) Ann. N. Y. Acad. Sci. 777: 332-337).

Consequently, protein kinases and phosphatases have become attractive targets for therapeutic intervention, and many clinically useful drugs have been designed, or have been found to act on protein kinases or phosphatases. Strategies employing kinase inhibitors for the treatment of cancer have recently been extremely successful. For example, Gleevec, which inhibits BCR-Abl has had great success in the treatment of CML (REFS). In addition, a small molecule inhibitor of the EGFR, Iressa, has recently been approved for lung cancer. In other examples, various protein kinase C inhibitors and inhibitors of cyclin dependent kinase are in clinical trials as therapeutic agents for the treatment of cancer (Clin. Cancer Res. (1995) 1:113-122), and J. Mol. Med. (1995) 73:(10):509-14).

Interestingly, recent research indicated that previous studies grossly underestimated the number of different protein kinases involved in signaling. Thus, numerous new potential drug targets may become available. However, with an increasing number of kinases as targets for inhibitors, new difficulties have become readily apparent. Among other problems, many of the known methods for measuring protein phosphorylation rely on the incorporation and measurement of ³²P or ³³P into a protein substrate of interest. Unfortunately, the level of incorporation of radioisotopes into target proteins is often very low, rendering radioisotope assays relatively insensitive. Another problem in these experiments is the promiscuity of the kinase for non-physiological substrates. Consequently, radioisotope based assays will often not provide useful data on preferential specificity of an inhibitor against multiple kinases.

To improve specificity in such assays, phosphorylation-specific antibodies may be employed in an ELISA-type approach to detect the phosphorylated substrate (which may or may not be labeled). However, depending on the substrate, a proper antibody may not be available or may be difficult to obtain. Moreover, such alternative methods nevertheless require cell lysis, multiple incubation and washing stages and further sample processing, which is generally time consuming, difficult to automate, and potentially susceptible to artifacts.

Numerous alternative non-radioisotope based assays (e.g., HPLC/MS, isothermal titration calorimetry, surface plasmon resonance, fluorescence polarization, etc. tests) are described in Protein Kinase Protocols by Alastair D. Reith (Humana Press; ISBN: 0896037002) or in Protein Phosphorylation: A Practical Approach by D. Grahame Hardie (The Practical Approach Series, No 123; Oxford University Press; ISBN: 0199633053), both of which are incorporated by reference herein. However, most of the known assays for protein phosphorylation identify or test single kinases in a cell free and highly artificial system. Moreover, such assays frequently require tedious sample processing steps and will therefore present significant obstacles towards reliable high-throughput systems (e.g., relatively high inter-sample and intra-sample variation due to numerous processing steps).

To overcome problems associated with radioisotope or phosphate incorporation into a target and to adapt a test to a high throughput cell-based assay, a cell of interest can be transfected with a reporter gene that is expressed under the control of a promoter responsive to a particular signaling pathway as described in U.S. Patent Application U.S. 2002/0142287 to Yamamoto et al., which is incorporated by reference herein. In this example, the inventors tested various compounds by exposing cells with the compounds in a high throughput screening assay format and observed bioluminescence of the cells in response to those compounds.

While such systems tend to increase sensitivity and the number of assays that can be performed, various disadvantages still remain. For example, such systems generally do not allow differentiation of inhibitory activity of the compound between various kinases within a particular pathway (i.e., no intra-pathway specificity). Moreover, adverse effects of the compound against a kinase specified for a pathway not controlling the reporter gene may not be identified (i.e., no inter-pathway specificity). Furthermore, a compound that may be cytotoxic and indirectly affect a kinase activity will be scored as an inhibitor. Still further, Yamamoto's system is generally limited to the particular set of kinases of the cell chosen. Consequently, if a particular kinase of a particular pathway is suspected in a particular cell type, that particular cell line needs to be engineered to include the reporter gene under the appropriate control.

Thus, although various methods and systems for kinase assays are known in the art, all or almost all of them suffer from one or more disadvantages. Therefore, there is still a need to provide improved systems and methods for testing kinase inhibition.

### SUMMARY OF THE INVENTION

The present invention is directed to systems and methods of testing kinase inhibition and particularly to those systems and methods allowing identification and characterization of novel kinase inhibitors. Contemplated systems and methods may further be employed for analysis of inter-pathway and/or intra-pathway inhibition of a kinase inhibitor, and may still further provide information on inhibition specificity of a particular kinase inhibitor.

In one aspect of the inventive subject matter, a method of kinase inhibition profiling includes one step in which simultaneously a first data set having a first plurality of data elements and a second data set having a second plurality of data elements are generated, wherein the first data set is associated with kinase inhibition in a first kinase signaling pathway, wherein the second data set is associated with kinase inhibition in a second kinase signaling pathway, and wherein each data element in the first and second data sets corresponds to an inhibition result of a kinase in the first and second kinase signaling pathways, respectively. In another step, at least one of the data elements from the first data set is used as reference data against at least one of the data elements from the second data set to calculate a normalized inhibition profile. Among other advantages, it should be recognized that the inhibition profile is normalized because all assay cell lines express the same recombinant reporter gene and all assay cell lines are derived from the same parent cell line so that any difference in kinase inhibition is not due to differences in reporter systems or differences in cellular absorption or metabolism.

In particularly preferred aspects of such methods, the step of using at least one of the data elements comprises using each of the data elements from the first data set as reference data against each of the data elements from the second data set to calculate the normalized inhibition profile, most preferably wherein the inhibition result is acquired in vivo from a cell that expresses from a stably transfected gene and in response to an inducer the kinase in a catalytically active form. In further preferred aspects, it is contemplated that the inhibition of the kinase alters a signal effected by a recombinant reporter gene, and wherein the recombinant reporter gene is functionally controlled by a transcription factor that is activated by a component of at least one of the first and second kinase signaling pathways.

In another aspect of the inventive subject matter, a method of analyzing intra-pathway kinase inhibition of a pharmaceutically active compound in a kinase signaling pathway has one step in which a plurality of cells (most preferably mammalian cells, e.g., 293 cells) is provided that express from a stably transfected gene, and in response to an inducer (e.g., doxycycline), a plurality of kinases in a catalytically active form, wherein the plurality of cells further express a reporter gene (e.g., gene encoding luciferase) in response to catalytic activity of the kinases, and wherein the reporter gene in each of the plurality of cells is the same (and wherein the plurality of cells are derived from a single cell type, wherein a first kinase of the plurality of kinases is different from a second kinase of the plurality of kinases, plurality of kinases, and wherein the first and second kinases are members of the same kinase signaling pathway). In another step, the plurality of cells is simultaneously induced with the inducer, and the cells are simultaneously presented with the pharmaceutically active compound. In a still further step, a first signal effected by the reporter gene is acquired from a cell expressing the first kinase, and a second signal effected by the reporter gene is acquired from a cell expressing the second kinase. In yet another step, a kinase is identified within the kinase signaling pathway as being inhibited by the pharmaceutically active compound using the first and second signals, wherein the first signal is normalized using the second signal (typically, the first signal is compared to the second signal).

While not limiting to the inventive subject matter, it is generally preferred that the catalytically active form of the kinase is a constitutively active kinase mutant or an autophosphorylating kinase, and/or that the reporter gene is expressed in response to activation of a transcription factor, wherein the activation is effected by a kinase within the kinase signaling pathway. Particularly preferred kinase signaling pathways include the MEK-ERK mediated pathway, the IKK-NFkB mediated pathway, the p38 mediated pathway, the JNK-Jun mediated pathway, the JAK-STAT mediated pathway, and the PI-3-Kinase/Akt pathway.

In a further aspect of the inventive subject matter, a method of analyzing inter-pathway inhibition of a pharmaceutically active compound in a first kinase signaling pathway and a second kinase signaling pathway may include one step in which a plurality of cells is provided that express from a stably transfected gene and in response to an inducer a plurality of kinases in a catalytically active form, respectively, wherein the plurality of cells further express a reporter gene in response to catalytic activity of the kinases, and wherein the reporter gene in each of the plurality of cells is the same (wherein the plurality of cells are derived from a single cell type, wherein a first kinase of the plurality of kinases is a member of a first kinase signaling pathway, and wherein a second kinase of the plurality of kinases is a member of a second kinase signaling pathway). In a further step, the plurality of cells is simultaneously induced with the inducer, and the cells are simultaneously presented with the pharmaceutically active compound. In a further step, a first signal effected by the reporter gene is acquired from a cell expressing the first kinase, and a second signal effected by the reporter gene is acquired from a cell expressing the second kinase. In a still further step, a kinase is identified within the first and second kinase signaling pathways as being inhibited by the pharmaceutically active compound using the first and second signals, wherein the first signal is normalized using the second signal. With respect to the cells, kinases, signaling pathways, inducers, reporter genes, etc., the same considerations as described above apply.

Therefore, a high-throughput screening system may include a plurality of cells that express from a stably transfected gene and in response to an inducer a plurality of kinases in a catalytically active form, respectively, and wherein the plurality of cells further express a reporter gene in response to catalytic activity of the kinases (wherein the plurality of cells are derived from a single cell type, and wherein cells expressing a first kinase of the plurality of kinases are separate from cells expressing a second kinase of the plurality of kinases). An acquisition system acquires a first signal from the cells expressing the first kinase and a second signal from the cells expressing the second kinase, wherein first and second signals are effected by the reporter gene, and a data processor identifies a kinase as being inhibited by a pharmaceutically active compound using the first and second signals. Again, with respect to the cells, kinases, signaling pathways, inducers, reporter genes, etc., the same considerations as described above apply.

Various objects, features, aspects and advantages of the present invention will become more apparent from the following detailed description of preferred embodiments of the invention, along with the accompanying drawings in which like numerals represent like components.

### BRIEF DESCRIPTION OF THE DRAWINGS

FIG. 1 is a schematic of an exemplary IKK2 pathway specific reporter assay.

FIG. 2 is a schematic of an exemplary MEK-ERK pathway specific reporter assay.

FIG. 3 is a schematic of a cell depicting multiple signal transduction pathways.

FIG. 4 is a schematic of one signal transduction pathway of FIG. 3.

FIG. 5 is an autoradiograph showing expression of MEK and MEK* in response to induction with doxycycline.

FIG. 6 is an autoradiograph showing effects of MEK and MEK* expression on ERK phosphorylation.

FIG. 7 is a bar graph showing luminescence from an exemplary assay cell line in response to induction and incubation with various inhibitors.

FIG. 8 is a table showing intra and inter pathway results from incubation of assay cells with various inhibitors in a MAPK pathway screening assay.

FIG. 9 is a western-blot showing expression of luciferase in response to induction with doxycycline.

FIG. 10 is a scattergram representing exemplary test results from a test system with a MEK cell set and an IKK2 cell set.

### DETAILED DESCRIPTION

The inventors discovered that a parallel inducible cell-based kinase screen (PICKS) can be established to identify novel kinase inhibitors, inhibition specificity of a particular kinase inhibitor, and analysis of inter-pathway and/or intra-pathway inhibition of a kinase inhibitor. Contemplated systems generally include a plurality of cells that express a kinase gene under the control of an inducer, and a reporter gene, wherein the cells are derived from a single cell line, and wherein the reporter gene generates a signal in response to catalytic activity or inhibition of the expressed kinases. In particularly preferred systems, the cells express kinases from a kinase family, kinases from a single kinase signaling pathway, and/or kinases from various different kinase signaling pathways. In the most preferred systems, a plurality of cells that express a plurality of kinases in a single cell type with signals of the same reporter gene are presented to the test compounds to enable normalized inhibition profiles to be obtained.

Contemplated Cells

In an especially preferred aspect of the inventive subject matter, 293 cells (permanent line of primary human embryonal kidney, e.g., ATCC number CRL-1573) are employed to create founder cells. The term “founder cell” as used herein refers to a cell from which the assay cells are prepared. Most typically, founder cells will include a reporter gene but not a recombinant kinase gene of interest (i.e., a-gene encoding a kinase in addition to the kinase genes naturally occurring in the founder cell). Thus, the term “assay cell” refers to a founder cell that includes a recombinant kinase gene of interest. Most typically, assay cells will include both the reporter gene and the recombinant kinase gene.

However, in alternative aspects of the inventive subject matter numerous alternative founder cells are also contemplated. For example, where metabolic conversion of a test compound in an assay cell is likely or desired, hepatic founder cells may be especially suitable (e.g., Hep G2 human hepatocellular carcinoma cells, commercially available as ATCC HB-8065). In another example, where a test compound may exhibit activity against diseased cells, neoplastic or otherwise, replication defect cells may be employed (e.g., human pancreatic carcinoma cells, commercially available as ATCC HTB-134). In still further contemplated aspects, cells with known kinase dysfunctions (e.g., overexpressed, mutated, truncated, etc.; e.g., slight constitutive activation of the MAP kinase and MEK proteins in human bone marrow cells, commercially available as ATCC CRL-2541) may be employed.

It is generally preferred that the founder cells (and consequently also the assay cells) are mammalian cells, which may be derived from healthy or diseased human cells, or where the founder cells are non-mammalian cells that the founder cells can provide a model for mammalian cells, and especially human cells. Thus, and depending on the cell type and the desired characteristics of the founder and assay cells, suitable founder and assay cells may be primary cell lines from a donor, established cell lines from commercially available cells, or cell lines propagated from commercially available stem cells. Therefore, it should be recognized that the particular choice of founder cells or assay cells will depend at least in part on the particular assay, test compound, or other experimental conditions. However, it is generally required that the founder cell can be transformed to express (at least transiently, and more typically stably) the reporter gene and the recombinant kinase gene of interest. Among other sources, an exemplary collection of suitable human founder cells is available from the American Type Culture Collection (ATCC), P.O. Box 1549, Manassas, Va. 20108.

In yet further contemplated aspects of the inventive subject matter, it should be appreciated that founder cells and/or assay cells with at least one of a reporter gene and a recombinant kinase gene may be employed as functional controls (e.g., to determine proper induction), negative controls (e.g., to determine potential adverse effects of expressed recombinant kinase) and/or controls to assess toxicity of the tested compounds.

Contemplated Reporter Genes

It is generally preferred that the reporter gene is a gene that is not naturally expressed in the founder cell, and that the gene product of the reporter gene provides directly or indirectly a quantifiable signal (e.g., optically, radiometrically, or electromagnetically identifiable signal), wherein the signal most preferably can be detected and quantified in vivo without processing (e.g., isolating and disintegrating) the assay cells. For example, preferred reporter genes encode an enzyme that converts a substrate to a luminescent product or precursor that leads to luminescence. A typical representative for such reporter genes is the gene encoding luciferase that converts luciferin with ATP (adenosine 5′-triphosphate) to oxyluciferin, CO₂, AMP (adenosine5′-monophosphate), and light. Alternatively, suitable reporters may also be enzymes that include chloramphenicol acetyltransferase, or enzymes that provide a signal via generation of a chromophore or chromophore precursor (beta-galactosidase hydrolyzing 5-bromo-4-chloro-3-indoyl-beta-D-galactopyranoside).

However, in further alternative (and less preferred) aspects, the reporter gene may also be a gene that is present and/or expressed in the founder cells. For example, suitable reporter genes include all enzymes that upon reaction with a natural or synthetic substrate will provide a quantifiable signal (e.g., light absorption/color change, luminescence, fluorescence, etc.), and it is particularly preferred that the quantifiable signal can be measured without processing (e.g., lysing, homogenizing, extracting, etc.) the assay cell.

It is generally preferred that the reporter gene is operationally coupled to a promoter, wherein the promoter is responsive to activation by an element of one or more kinase signaling pathways. The term “operationally coupled” as used herein means that the reporter gene is expressed in response to an activating event (e.g., binding of a transcription activator, release of a repressor, interaction with an enhancer or upstream activating sequence, etc.) on the promoter. Therefore, the term “promoter” as used herein encompasses all sequences that are involved in the expression of the reporter gene in response to the activating event, and particularly include a DNA-dependent RNA polymerase binding site, a DNA-dependent RNA polymerase transcription initiation site, repressor binding sites, transcription activator (e.g., transcription factor) binding sites, upstream activating elements, and enhancers.

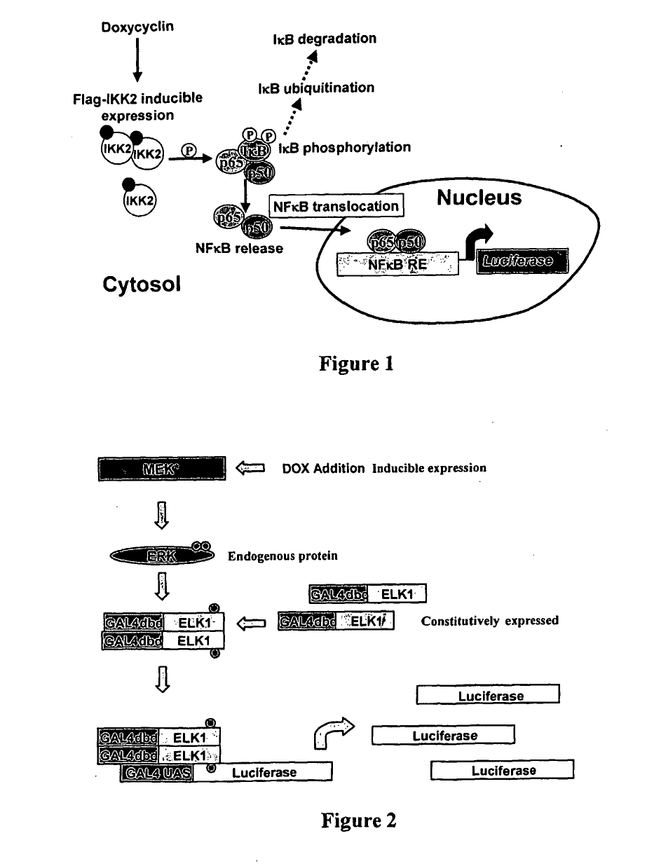

In particularly preferred aspects of the inventive subject matter, the promoter includes a sequence element that is activated by an element of a single kinase signaling pathway. For example, where the kinase signaling pathway is the IKK2 pathway, the promoter may comprise an NF-kB responsive element (see e.g., FIG. 1). In another example, where the kinase signaling pathway is the p38 pathway, the promoter may comprise an ATF2 binding/responsive sequence. Alternatively, the promoter may also include a sequence non-native to the founder cell, wherein the activating element may be a fusion protein with a component of one or more kinase signaling pathways, and wherein the fusion protein binds a respective binding sequence to activate expression of the reporter gene. For example, where the kinase signaling pathway is a MEK-ERK pathway, the recombinant fusion protein may comprise a GAL4 DNA binding domain and the ELK1 polypeptide of the MEK-ERK pathway. Upon phosphorylation and dimerization of the ELK1 domain, the two GAL4 DNA binding domains dimerize and consequently bind to the GAL4 upstream activating sequence to thereby activate expression of the reporter gene (see FIG. 2).

In still further contemplated aspects, it should be recognized that the reporter gene may be also coupled to additional functional sequence elements, wherein the additional functional sequence elements may improve expression rate or catalytic behavior, prevent precipitation or export from the assay cell, etc. For example, the reporter gene may include one or more internal ribosomal entry sites, or affinity tags (e.g., His₆-tag).

Reporter genes may also be represented by fusion proteins of luciferase and specific signaling modules from endogenous pathways. For example, a chimeric protein that expresses luciferase and that also contains the sequences through which IkB is phosphorylated by IKK2 and is subsequently ubiquitinated and destroyed. A similar reporter gene can be used for Wnt/β-catenin signaling, which is a signaling molecule also regulated by ubiquitin-mediated destruction. Therefore, in these cases, inhibiting the target kinases will result in an elevated luminescent signal.

Transfection of the cells to establish a cell line that stably includes the reporter gene (under the control of suitable promoter elements) may be performed using numerous methods well known in the art, and a particular method of Transfection will typically depend on the particular founder cell line as well as on further particular considerations. For example, where the founder cell line is relatively robust, cells may be transfected via electroporation or calcium phosphate DNA precipitation. Alternatively, lipofection or viral transfection may be employed for more sensitive cells. Consequently, it should be recognized that the transfected founder cell may include the reporter gene integrated into the genome of the founder cell, or that the reporter gene and promoter may be present in an extrachromosomal construct (e.g., artificial chromosome, plasmid, viral DNA/RNA, etc.). Therefore, the number of copies of the reporter and promoter genes may vary substantially.

Contemplated Kinases

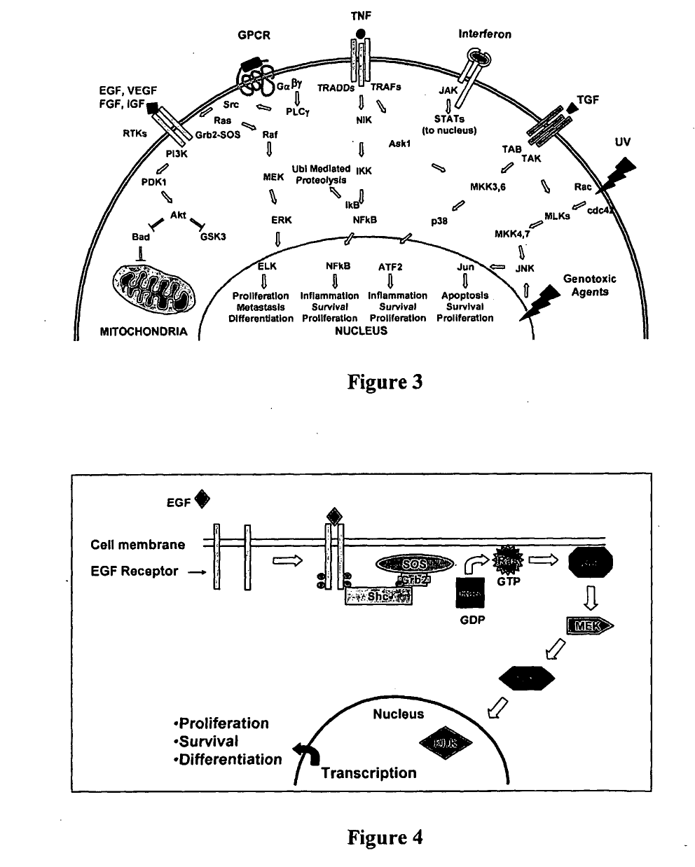

In a particularly preferred aspect of the inventive subject matter, suitable kinases include all kinases that are elements in kinase signaling pathways. The term “kinase signaling pathway” as used herein refers to a plurality of components in a cell that functionally cooperate to transmit an extracellular signal received by the cell to the nucleus (or other compartment) of the cell. Particularly contemplated extracellular signals include cytokines (e.g., IFN-alpha/beta/gamma, IL-12, IL-2, TNF-alpha), growth factors (e.g., EGF, VEGF, FGF, TGF, etc.), non-biological stimuli (e.g., UV radiation, reactive oxygen species, etc.), and signals for G Protein coupled receptors (GPCR ligands). An overview of exemplary kinase signaling pathways is given in FIG. 3 (with a more detailed view of the MEK pathway in FIG. 4), and further contemplated exemplary kinases in kinase signaling pathways are described in the “Protein Kinase Facts Book” by Grahame Hardie and Steven Hanks (Academic Press; ISBN: 0123247195).

Thus, particularly preferred kinases include all kinases that are functional elements in the MEK-ERK mediated pathway (e.g., raf, MEK, ERK), the IKK-NFkB mediated pathway (e.g., TAK, NIK, IKK), the p38 mediated pathway (e.g., MKK3, MKK6, p38), the JNK-Jun mediated pathway (e.g., MLK, MKK4, MKK7, JNK), the JAK-STAT mediated pathway (e.g., JAK1-3, TYK2, and STATS 1-6), and/or the lipid mediated PI-3-Kinase pathway (e.g. PI4,5-Kinase, PTEN, PI-3-Kinase, PDK1, Akt1-3, and GSK3).

Still further, it is also preferred that suitable kinases are present in the assay cell in a constitutively active form. For example, introduction of acidic residues to particular positions in MEK (218 and 222 in MEK1), or deletion of certain domains in Akt or Raf (see e.g., Alessi et al., Curr. Biol. (1997), 7, 776-89; or Chou et al., J. Biol. Chem. (1995), 270(23), 14100-6; or Cohen et al., FEBS Lett. (1997), 410 (1), 3-10) can cause activation of the selected kinase without input from upstream elements. Consequently, it is contemplated that the genes encoding the kinases according to the inventive subject matter may be modified (e.g., via point mutations, insertions or deletions) to generate a constitutively active form. Alternatively, suitable kinases may also be autophosphorylating (i.e., self-activating) kinases, and include, for example, the Kit/SCFR or PDGF receptor. Such constitutively active forms advantageously provide a system in which no “upstream” activation of the kinase is required. Alternatively, “upstream” activating elements may be added to the assay cell or cotransfected with the kinase expression cassette where a particular kinase is not constitutively active.

Contemplated kinases are preferably expressed from a recombinant construct that includes the gene encoding the respective kinase and a promoter that controls/initiates transcription/expression of the desired kinase. With respect to the particular choice of an expression cassette it should be recognized that all expression cassettes are deemed suitable for use herein. There are numerous (commercially available) expression cassettes known in the art, and all of the known expression cassettes are contemplated herein. Thus, suitable expression cassettes comprising a promoter and the kinase gene on artificial chromosomes, plasmids, viral genomes, all of which may or may not be integrated into the genome of the assay cell. Thus, the expression of suitable kinases may or may not be inducible, and may or may not be stable (as opposed to transient).

However, in especially preferred aspects of the inventive subject matter, the expression cassette is stably transfected into the assay cell genome and expression of the kinase of interest is under the control of an inducible promoter, wherein the promoter does not naturally occur in the assay cell. For example, expression may be driven from the commercially available Tet-On/Off expression system (e.g., by Invitrogen), which employs a chimeric transactivator to regulate transcription of the gene of interest (here: the kinase of interest) from a silent promoter. The transactivator, either tTA or rtTA, is expressed from a constitutive CMV promoter present in the expression cassette (alternatively, the transactivator can also be expressed from a tissue-specific promoter). In the BD Tet-On™ System, rtTA binds to the TRE (Tetracycline Response Element) and activates transcription in the presence of doxycycline, which is a synthetic analog of Tetracycline. Without doxycycline, substantially no recombinant protein is expressed.

It should be particularly appreciated that inducible and regulated kinase expression systems in contemplated assay methods will provide numerous advantages over currently employed cell-based systems. Among other things, induction of expression of a constitutively active kinase will prevent toxic effects precipitated by non-induced expression, which will significantly reduce the selection pressure on the recombinant assay cells. Furthermore, providing relatively high levels of the recombinant kinase of interest, the signal due to inhibition of the kinase by a test compound will be a significant improvement over an endogenous kinase background.

Thus, particularly contemplated assay cells will (a) be derived from a single founder cell or single founder cell line, (b) include a reporter gene under the control of a promoter that is responsive to activation by an element within a kinase signaling pathway, and (c) include a gene encoding a kinase of interest, where in the kinase of interest is preferably in a constitutively active form.

Analysis of Intra-Pathway Kinase Inhibition

In one particularly contemplated aspect of the inventive subject matter, the inventors have discovered that a plurality of assay cells comprising a reporter gene and a plurality of kinase genes of interest, respectively, may be employed to analyze intra-pathway kinase inhibition of a pharmaceutically active compound in a kinase signaling pathway.

For example, a plurality of cells may be constructed to express from a stably transfected gene and in response to an inducer a plurality of kinases in a catalytically active form, respectively, wherein the plurality of cells further express a reporter gene in response to catalytic activity of the kinases, and wherein the reporter gene in each of the plurality of cells is the same.

It is especially preferred that the assay cells are derived from human 293 founder cells (commercially available), wherein the 293 cells are transformed with an expression cassette comprising the gene encoding luciferase as a reporter gene, and wherein expression of the luciferase is under the control of the ELK binding sequence as promoter element of the MEK-ERK kinase signal transduction pathway.

A first portion of the so transformed founder cells is then transformed with a second expression cassette to provide inducible expression of MEK* (constitutively active form of MEK) as the first kinase of the MEK-ERK kinase signal transduction pathway. Similarly, a second portion of the transformed founder cells is transformed with another second expression cassette to provide inducible expression of ERK* (constitutively active form of ERK) as the second kinase of the MEK-ERK kinase signal transduction pathway. Preferred expression cassettes for the kinases are Tet-On™ expression cassettes as the expression of the kinases can be induced via doxycycline.

It should be recognized that in such double transformed assay cells (i.e., cells transformed with reporter gene and kinase gene, both with respective promoters), the expressed and constitutively active kinase will provide downstream activation (where appropriate) of other kinases of the same kinase signaling pathway and eventually lead to activation of the promoter of the luciferase gene, and thereby to an optically detectable signal.

Since the plurality of assay cells expressing MEK and ERK kinases are derived from a single cell type (here: the founder 293 cells), the optically detectable signal is normalized against the cell line. Moreover, as the first kinase of the plurality of kinases (here: the MEK kinase) is different from a second kinase of the plurality of kinases (here: the ERK kinase), the results provided are specific for the same kinase signaling pathway (here: the MEK-ERK pathway). Thus, it is generally preferred that an intra-pathway assay will employ a high-throughput screening format in which the assay cells are simultaneously induced with the inducer, and in which the assay cells are simultaneously presented with a test compound (e.g., a pharmaceutically active compound). The term “simultaneously induced” as used herein means that all of the tested cells are incubated with the inducer (or exposed to inducing conditions) within a period of less than one hour, more typically less than 15 minutes, and most typically less than 5 minutes. For example, in some preferred aspects of the inventive subject matter, the cells are simultaneously induced and treated with the test compound over a period of 24 hours. In most of such experiments, cells are trypsinized, counted, appropriately diluted, and the inducer added. These cells are then immediately plated on compound containing plates, usually within one hour of induction.

Acquisition of the signal from the cells previously induced with the inducer and presented with the test compound may vary considerably, and a particular method of acquisition will typically depend on the particular signal provided by the cell. However, it is generally preferred that acquisition will be integrated into a high-throughput system, and most preferably comprise luminometry (e.g., determination of luminescence, fluorescence, or phosphorescence) or spectrometry (e.g., light absorption at particular wavelength). In a particularly preferred aspect, acquisition of the signal will comprise (a) acquiring a first signal effected by the reporter gene from an assay cell expressing the first kinase and (b) acquiring a second signal effected by the reporter gene from an assay cell expressing the second kinase. Thus, the data acquisition is not only normalized against the cell type, but also against the same assay conditions and signal generation/acquisition system. Consequently, a kinase (here: the MEK or ERK kinase) can be identified within the kinase signaling pathway (here: the MEK-ERK pathway) as being inhibited by the test compound using the first and second signals, wherein the first signal is normalized using the second signal.

Of course it should be recognized that the founder cells in contemplated assays need not be limited to human 293 kidney cells, and it should be appreciated that all cells that can express the kinase and the reporter gene under the control of their respective promoters may be suitable for use herein. Consequently, appropriate cells particularly include human cells (which may or may not be diseased), and cells that provide a model for human cells. Further, generally and especially contemplated cells are those detailed in the section entitled “Contemplated Cells” above.

Similarly, it should be recognized that numerous alternative kinases from various kinase signaling pathways other than the MEK-ERK pathway are also contemplated, and especially suitable kinases from alternative pathways include those associated with the IKK-NFkB mediated pathway, p38 mediated pathway, JNK-Jun mediated pathway, PI3-Kinase-Akt mediated pathway, and JAK-STAT mediated pathway. Furthermore, and as already discussed above, it is generally preferred that the kinase that is expressed in the assay cell is catalytically active (e.g., as a constitutively active kinase mutant or an autophosphorylating kinase). Depending on the expression cassette for the kinase of interest, it should be recognized that induction may vary considerably. For example, suitable promoters controlling expression of the kinase gene may be activated by de-repression, binding of one or more transcription factors, temperature changes, etc. However, it is generally preferred that the induction is performed with doxycycline. Similarly, it is preferred that the reporter gene is expressed in response to activation of a transcription factor, and wherein the activation is effected by a kinase within the kinase signaling pathway.

Analysis of Inter-Pathway Kinase Inhibition

In another particularly contemplated aspect of the inventive subject matter, the inventors have discovered that a plurality of assay cells comprising a reporter gene and a plurality of kinase genes of interest, respectively, may be employed to analyze inter-pathway kinase inhibition of a pharmaceutically active compound in a first kinase signaling pathway and a second kinase signaling pathway.

For example, a plurality of cells may be constructed that express from a stably transfected gene and in response to an inducer a plurality of kinases in a catalytically active form, respectively, wherein the plurality of cells further express a reporter gene in response to catalytic activity of the kinases, and wherein the reporter gene in each of the plurality of cells is the same. With respect to the cells and their construction, the same considerations as described above apply (e.g., the assay cells are derived from a single cell type). However, it should be recognized that in such assays a first kinase of the plurality of kinases (e.g., MEK) is a member of a first kinase signaling pathway (e.g., MEK-ERK pathway), and that a second kinase (e.g., NIK) of the plurality of kinases is a member of a second kinase signaling pathway (e.g., IKK-NF-kB pathway).

Since the plurality of assay cells expressing MEK and NIK kinases are derived from a single cell type (here: the founder 293 cells) with a common reporter, the optically detectable signal is normalized against the cell line with respect to response of the activated reporter and the response of the cell to the pharmaceutically active compound in terms of absorption and metabolism. Moreover, as the first kinase of the plurality of kinases (here: the MEK kinase) is different from a second kinase of the plurality of kinases (here: the NIK kinase) and belongs to a different kinase signaling pathway, the results provide specific inhibition data for the different kinase signaling pathway (here: the MEK-ERK pathway versus IKK-NFkB pathway). Thus, it is again generally preferred that an inter-pathway assay will employ a high-throughput screening format in which the assay cells are simultaneously induced with the inducer, and in which the assay cells are simultaneously presented with a test compound (e.g., a pharmaceutically active compound).

Consequently, such contemplated methods will include a step of simultaneously inducing the plurality of cells with the inducer, and simultaneously presenting the cells with the pharmaceutically active compound, and another step of acquiring a first signal effected by the reporter gene from a cell expressing the first kinase and acquiring a second signal effected by the reporter gene from a cell expressing the second kinase. In a still further step, a kinase can be identified within the first and second kinase signaling pathways as being inhibited by a test compound using the first and second signals, wherein the first signal is normalized using the second signal. Again, with respect to the cells, inducers, kinases, reporter genes, kinase signaling pathways, and other elements common with the intra-pathway analysis as described above, the same considerations apply.

Kinase Inhibition Profiling

In a still further aspect of the inventive subject matter, the inventors contemplate a method of kinase inhibition profiling in which the specificity of a kinase inhibitor against one or more kinases of the same or different kinase signaling pathway can be readily determined.

In such contemplated methods it is generally preferred that a first data set having a first plurality of data elements is simultaneously generated with a second data set having a second plurality of data elements, wherein the first data set is associated with kinase inhibition in a first kinase signaling pathway, wherein the second data set is associated with kinase inhibition in a second kinase signaling pathway, and wherein each data element in the first and second data sets corresponds to an inhibition result of a kinase in the first and second kinase signaling pathways, respectively. At least one of the so obtained data elements from the first data set may then be employed as reference data against at least one of the data elements from the second data set to calculate a normalized inhibition profile. It should be understood that the first data set need not be generated at the same time as the second data set and that comparisons will be made to more than one data set.

In particularly preferred aspects of the inventive subject matter, the first data set may be generated from one or more incubations of contemplated assay cells expressing a first kinase with a test compound, while the second data set may be generated from one or more incubations of contemplated assay cells expressing a second kinase with a test compound. Contemplated data sets may further be generated using the same concentration of the test compounds or increasing concentrations of the test compound to obtain saturation or other kinetic relevant data of the test compound relative to the kinase of interest. Simultaneous generation of the data sets is typically performed using a high-throughput assay format in which the assay cells are simultaneously induced, and later simultaneously presented with the test compound. Furthermore, it is generally preferred that acquisition of the signal is also performed simultaneously (see also above). Thus, a data element typically represents a single inhibition result (or average from multiple inhibition results) from a single test compound with a single type of assay cell (e.g., assay cells expressing MEK).

In further particularly preferred aspects, it is contemplated that the step of using at least one of the data elements comprises using each of the data elements from the first data set as reference data against each of the data elements from the second data set to calculate the normalized inhibition profile. For example, where the data elements of the first data set comprise inhibition data of a first kinase with a test compound at increasing concentration, each of the data elements may be employed to normalize results for inhibition of a second kinase with the same inhibitor at the same increasing concentrations.

Inhibition results are preferably acquired in vivo from cells that express from a stably transfected gene and in response to an inducer a kinase of interest in a catalytically active form (see above), wherein inhibition of the kinase alters a signal effected by a recombinant reporter gene, which is preferably functionally controlled by a transcription factor that is activated by a component of at least one of the first and second kinase signaling pathways (see above). Thus, preferred test compounds are generally all compounds that exhibit inhibition of a kinase to at least some degree, and it should be appreciated that suitable test compounds may be individual compounds or library compounds.

In yet further contemplated aspects of the inventive subject matter, it should also be appreciated that contemplated assays may be performed in a multiplexing or co-culturing approach. In such methods and configurations, multiple and distinct assay cells are tested in a single well (or other test vessel) at the same time under identical culture conditions. Among other things, such methods may be particularly advantageous where inhibition data are highly variable on culture conditions. Alternatively, such multiplex assays may combine multiple kinases of one pathway in a single test. On the other hand, where desirable, it is also contemplated that assays may be set up in a manner such that multiple compounds are exposed to a single (or multiple) type of assay cell in a single well (or other test vessel). For example, a multi-well test plate may be set up in which combinations of four compounds per well are tested against a single type of assay cell, and wherein each plate has four different arrays.

Therefore, a high-throughput screening system may comprise a plurality of cells that express from a stably transfected gene and in response to an inducer a plurality of kinases in a catalytically active form, respectively, and wherein the plurality of cells further express a reporter gene in response to catalytic activity of the kinases. In most preferred systems, the plurality of cells is derived from a single cell type, and the cells expressing a first kinase of the plurality of kinases are separate from cells expressing a second kinase of the plurality of kinases. An acquisition system (e.g., plate reader for luminescence or absorption) acquires a first signal from the cells expressing the first kinase and a second signal from the cells expressing the second kinase, wherein first and second signals are effected by the reporter gene, and a data processor identifies a kinase as being inhibited by a pharmaceutically active compound using the first and second signals. It should further be appreciated that in contemplated screening systems, the first and second kinases may belong to the same kinase signaling pathway or to different kinase signaling pathways, and the same considerations for the kinases and kinase pathways as described above apply. With respect to the cells, the reporter genes and the acquisition system, the same considerations as discussed above apply.

Furthermore, it should be appreciated that by employing a cell based and normalized test system, biologically relevant inhibition data (e.g., data that reflect transport of test compounds into a cell, metabolic conversion of a test compound in a cell, etc) may be generated. Moreover, by employing a high-throughput format (e.g., 384 wells per plate in a microtiter-plate based system), libraries of test compounds can be examined in a relatively short time, and multiple assay cells expressing multiple kinases (from the same or different kinase signaling pathway, or from the same kinase subfamily or different kinase subfamilies) may be tested simultaneously or separately in a normalized environment.

### EXAMPLES

In an exemplary and generalized outline of contemplated screening methods, a cell line is transformed with a reporter gene under the control of an element (e.g., transcription factor) of a kinase signaling pathway to generate the founder cell, and some founder cells that now include the reporter gene are further transformed with a gene encoding a first kinase (e.g., belonging to the MEK-ERK kinase signaling pathway). Other founder cells that now contain the reporter gene are transformed with a gene encoding a second kinase (also belonging to the MEK-ERK kinase signaling pathway).

Both resulting assay cells (designated K1 and K2) are exposed to a plurality of test compounds (potential kinase inhibitors), and the kinase-driven signal readout is measured with a detector such as a luminometer. The founder cells can be employed as further control, and toxicity, specificity and activity of the potential kinase inhibitors may then be determined in a normalized manner (here: data normalized via parallel experimental set up, normalized against the same cellular background and the same signal readout of the reporter, and further using same control cells).

### Stable Expression of MEK and AFEK* in 293 Cells

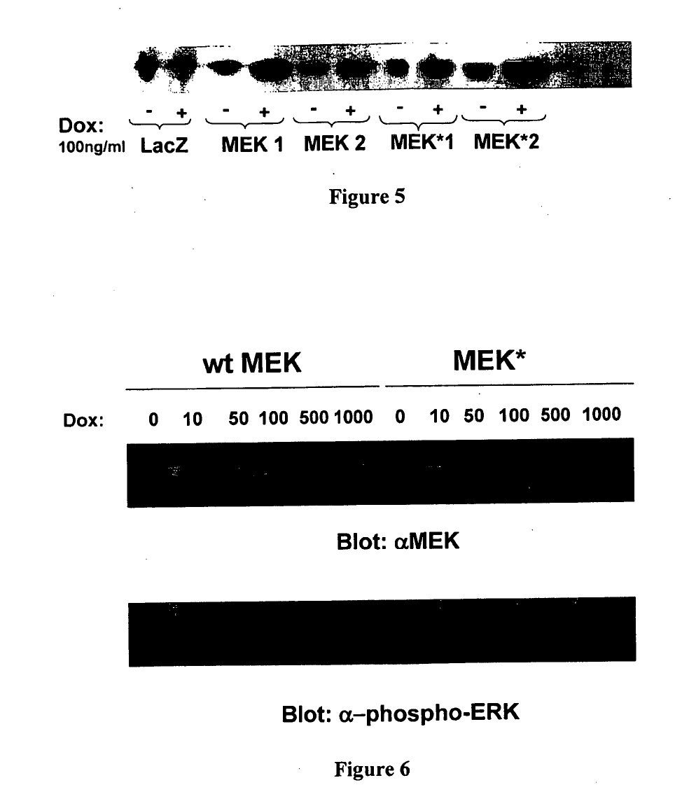

Cells that contain inducible wild type or mutant MEK (referred to as MEK*) were generated by transfecting 293T via lipid-mediated gene transfer of the following plasmids. Plasmids for the Tet repressor (Invitrogen pCDNA6-TR), Gal4-Elk1 (Stratagene), Gal4 UAS dependent luciferase (Stratagene), and Tet-On MEK or Tet-On MEK* (Invitrogen) were transfected into cells and the cells placed under selection in 500 ug/ml geneticin, 100 ug/ml zeocin, and 2.5 ug/ml blasticidin. Tet-On MEK was created by placing the cDNA sequence for human MEK under the control of the Tet-On promoter in pCDNA4TO (Invitrogen). Tet-On MEK* was created by site-directed mutagenesis of S218 and S222 to aspartic acids in the human MEK cDNA sequence. FIG. 5 depicts an autoradiograph (ECL) showing expression of MEK/MEK* in 293T cells.

### Effect of MEK and MEK* Expression on ERK Phosphorylation

Upon establishing Tet-On MEK and Tet-On MEK* clones, we tested the ability of doxycycline to induce gene expression that resulted in ERK phosphorylation and activation was tested. These clones were tested by plating cells from the respective clones and adding lug/ml doxycycline for the times indicated. Cells were lysed and the proteins run on an SDS-PAGE gel. Proteins were transferred to a membrane and the levels of ERK phosphorylation were detected by Western blotting with anti-phospho-ERK antibodies (Cell Signaling Technologies). The presence of phospho-ERK was visualized through attachment of a secondary antibody (goat-anti-rabbit) conjugated to alkaline phosphatase (AP). AP can cleave substrates thus releasing light to be captured on film or by a scanner as depicted in FIG. 6.

### Specificity of Assay Signals in MEK*Expressing Assay Cell Line

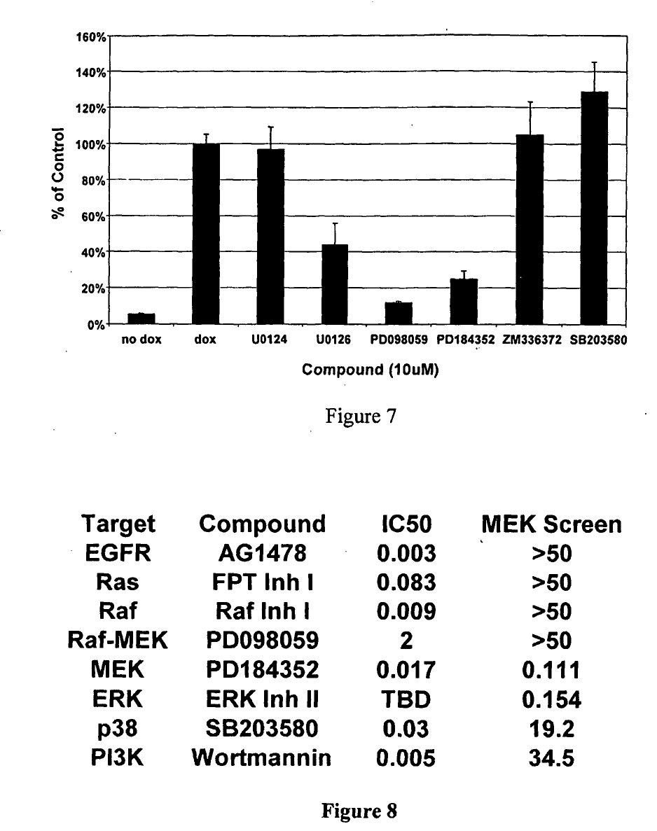

To test the specificity of the MEK* expressing cell lines, cells were trypsizned, counted, diluted to appropriate concentrations, induced with 100 ng/ml doxycycline, and plated onto compound containing plates. Compounds were used at 10 μM and the bars (FIG. 7) represent the average of three samples with standard deviation. U0126, PD098059, and PD184352 are MEK inhibitors. U0124 is a control for U0124. ZM336372 and SB203580 are Raf and p38 inhibitors, respectively. Cells were incubated for 24 hours. Steady-Glo luciferase reagent (Promega) was added and the plates read. Samples were standardized to internal positive and negative controls and the plates analyzed as depicted in FIG. 7.

### Selectivity and Specificity of Cell Based Assay with MEK*Expressing Cell Line

To determine the specificity and selectivity of the MEK* inducible cell line, cells were treated as above. Compounds used in these experiments were titrated from 50 μM down to 2.5 nM by three fold dilutions at each step. The target of each compound is indicated in FIG. 8. IC50 were from references for each compound. EC50s were generated by taking the average of each data point and analyzing the data with ExcelFit. The data indicate that only compounds at the level of MEK or downstream of MEK result in luciferase inhibition. Parallel pathways involving p38 and PI3 Kinase had little effect also. Therefore, the MEK* cell line is sensitive and selective for MEK or ERK inhibitors.

### Kinase Signaling Pathway-Dependent Reporter Gene Expression

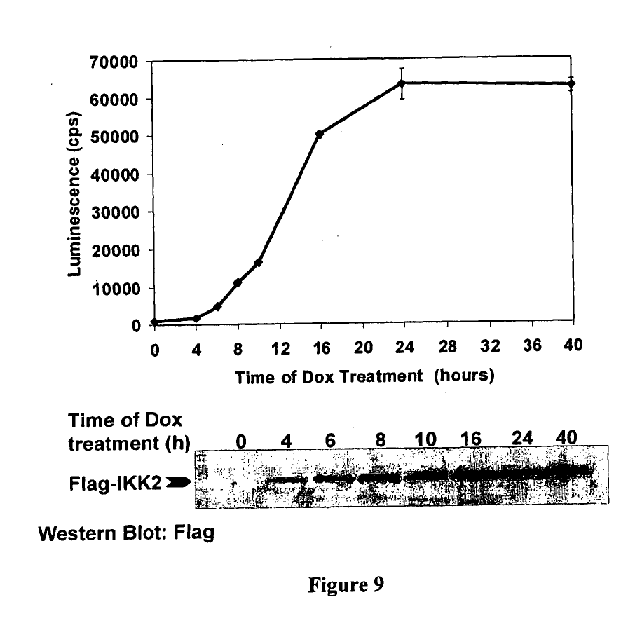

In FIG. 9, the expression of FLAG-tagged IKK2 is correlated with the increase in NFkB-mediated luciferase expression. Cells were trypsinized and plated, then treated with 100 ng/ml doxycycline. Samples were taken at the indicated times after induction and assayed by Western blot for IKK2 expression and using Bright-Glo (Promega) to determine luciferase expression.

### Data Analysis for Identification of Compounds with High Selectivity and Low Toxicity

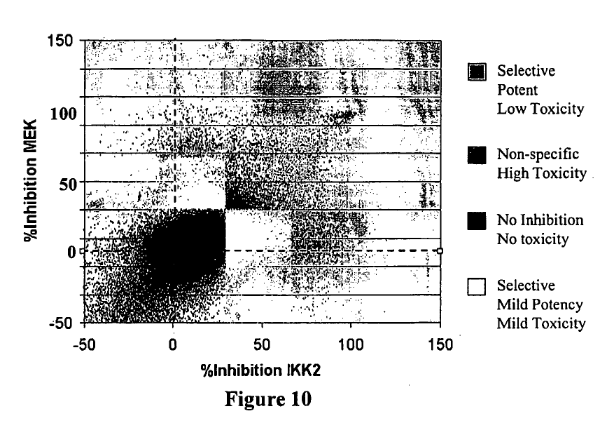

Data analysis may be performed in numerous manners, and it is contemplated that all manners of data presentation are considered suitable for use herein. However, it is generally preferred that multiple (i.e., at least two) sets of inhibition data, which may or may not be calibrated against a negative control, are graphically depicted in a scatter plot as shown in FIG. 10. Here, 600000 data points from a collection of 100,000 compounds were plotted against % inhibition, and predetermined cut-off yalues were used to color the corresponding data points falling within the ranges of the cut-off. In this example, inhibitor data points indicating kinase specific (here: inter-kinase pathway specific) and strong inhibition were marked green if the inhibition for the first kinase is between 75-100% and between 0-25% of the second kinase. Similarly, inhibitor data points indicating somewhat kinase specific and moderate inhibition were marked yellow if the inhibition for the first kinase is between 30-75% and between 30-75% of the second kinase. In contrast, where inhibition data points show inhibition that is greater than 30% for both kinases, data points were marked red to indicate no specificity and potential toxicity. Likewise, where inhibition data points show inhibition that is less than 30% for both kinases, data points were marked blue to indicate no inhibition and no potential toxicity.

Thus, specific embodiments and applications of parallel inducible cell-based kinase screens have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

What is claimed is:

1. A method of kinase inhibition profiling, comprising:
simultaneously generating a first data set having a first plurality of data elements, and a second data set having a second plurality of data elements;
wherein the first data set is associated with kinase inhibition in a first kinase signaling pathway, wherein the second data set is associated with kinase inhibition in a second kinase signaling pathway, and wherein each data element in the first and second data sets corresponds to an inhibition result of a kinase in the first and second kinase signaling pathways, respectively; and
using at least one of the data elements from the first data set as reference data against at least one of the data elements from the second data set to calculate a normalized inhibition profile.

2. The method of claim 1 wherein the step of using at least one of the data elements comprises using each of the data elements from the first data set as reference data against each of the data elements from the second data set to calculate the normalized inhibition profile.

3. The method of claim 2 wherein the inhibition result is acquired in vivo from a cell that expresses, from a stably transfected gene, and in response to an inducer, the kinase in a catalytically active form.

4. The method of claim 3 wherein inhibition of the kinase alters a signal effected by a recombinant reporter gene.

5. The method of claim 4 wherein the recombinant reporter gene is functionally controlled by a transcription factor that is activated by a component of at least one of the first and second kinase signaling pathways.

6. A method of analyzing intra-pathway kinase inhibition of a pharmaceutically active compound in a kinase signaling pathway, comprising:
providing a plurality of cells that express from a stably transfected gene and in response to an inducer a plurality of kinases in a catalytically active form, respectively, wherein the plurality of cells further express a reporter, gene in response to catalytic activity of the kinases, and wherein the reporter gene in each of the plurality of cells is the same;
wherein the plurality of cells are derived from a single cell type, wherein a first kinase of the plurality of kinases is different from a second kinase of the plurality of kinases, and wherein the first and second kinases are members of the same kinase signaling pathway;
simultaneously inducing the plurality of cells with the inducer, and simultaneously presenting the cells with the pharmaceutically active compound;
acquiring from a cell expressing the first kinase a first signal effected by the reporter gene and acquiring from a cell expressing the second kinase a second signal effected by the reporter gene; and
identifying a kinase within the kinase signaling pathway as being inhibited by the pharmaceutically active compound using the first and second signals, wherein the first signal is normalized using the second signal.

7. The method of claim 6 wherein the catalytically active form is a constitutively active kinase mutant or an autophosphorylating kinase.

8. The method of claim 7 wherein the reporter gene is expressed in response to activation of a transcription factor, and wherein the activation is effected by a kinase within the kinase signaling pathway.

9. The method of claim 8 wherein the kinase signaling pathway is selected from the group consisting of a MEK-ERK mediated pathway, a IKK-NFKB mediated pathway, a p38 mediated pathway, a JNK-Jun mediated pathway, a PI3K-Akt mediated pathway, and a JAK-STAT mediated pathway.

10. The method of claim 9 wherein the inducer is doxycycline and the reporter gene is a gene encoding luciferase.

11. The method of claim 10 wherein the cells are mammalian cells.

12. The method of claim 11 wherein the mammalian cells are 293 cells.

13. A method of analyzing inter-pathway inhibition of a pharmaceutically active compound in a first kinase signaling pathway and a second kinase signaling pathway, comprising:
providing a plurality of cells that express from a stably transfected gene and in response to an inducer a plurality of kinases in a catalytically active form, respectively, wherein the plurality of cells further express a reporter gene in response to catalytic activity of the kinases, and wherein the reporter gene in each of the plurality of cells is the same;
wherein the plurality of cells are derived from a single cell type, wherein a first kinase of the plurality of kinases is a member of a first kinase signaling pathway, and wherein a second kinase of the plurality of kinases is a member of a second kinase signaling pathway;
simultaneously inducing the plurality of cells with the inducer, and simultaneously presenting the cells with the pharmaceutically active compound;
acquiring from a cell expressing the first kinase a first signal effected by the reporter gene and acquiring from a cell expressing the second kinase a second signal effected by the reporter gene; and
identifying a kinase within the first and second kinase signaling pathways as being inhibited by the pharmaceutically active compound using the first and second signals, wherein the first signal is normalized using the second signal.

14. The method of claim 13 wherein the catalytically active form is a constitutively active kinase mutant or an autophosphorylating kinase.

15. The method of claim 14 wherein the reporter gene is expressed in response to activation of a transcription factor, and wherein the activation is effected by an element within at least one of the first and second kinase signaling pathways.

16. The method of claim 15 wherein the first and second kinase signaling pathways are selected from the group consisting of a MEK-ERK mediated pathway, a IKK-NFkB mediated pathway, a p38 mediated pathway, a JNK-Jun mediated pathway, a PI3K-Akt mediated pathway, and a JAK-STAT mediated pathway, and wherein the first and second kinase signaling pathways are not the same kinase signaling pathway.

17. The method of claim 16 wherein the inducer is doxycycline and the reporter gene is a gene encoding luciferase.

18. The method of claim 17 wherein the cells are mammalian cells.

19. The method of claim 18 wherein the mammalian cells are 293 cells.

20. A high-throughput screening system, comprising:
a plurality of cells that express from a stably transfected gene and in response to an inducer a plurality of kinases in a catalytically active form, respectively, and wherein the plurality of cells further express a reporter gene in response to catalytic activity of the kinases;
wherein the plurality of cells are derived from a single cell type, and wherein cells expressing a first kinase of the plurality of kinases are separate from cells expressing a second kinase of the plurality of kinases;
an acquisition system acquiring a first signal from the cells expressing the first kinase and a second signal from the cells expressing the second kinase, wherein first and second signals are effected by the reporter gene; and
a data processor that identifies a kinase as being inhibited by a pharmaceutically active compound using the first and second signals.

21. The screening system of claim 20 wherein the catalytically active form is a constitutively active kinase mutant or an autophosphorylating kinase.

22. The screening-system of claim 21 wherein the reporter gene is expressed in response to activation of a transcription factor, and wherein the activation is effected by an element within a kinase signaling pathway.

23. The screening system of claim 22 wherein the kinase signaling pathway is selected from the group consisting of a MEK-ERK mediated pathway, a IKK-NFKB mediated pathway, a p38 mediated pathway, a JNK-Jun mediated pathway, and a JAK-STAT mediated pathway, and wherein the first and second kinase signaling pathways are not the same kinase signaling pathway.

24. The screening system of claim 23 wherein the reporter gene is a gene encoding luciferase and the acquisition system comprises a luminometer.

25. The screening system of claim 20 wherein the first kinase of the plurality of kinases belongs to a kinase signaling pathway that is different from a kinase signaling pathway to which the second kinase of the plurality of kinases belongs.

26. The screening system of claim 20 wherein the first and second kinases of the plurality of kinases belongs to the same kinase signaling pathway.

27. A method of data processing, comprising:
providing a plurality of cells that express from a stably transfected gene and in response to an inducer a plurality of kinases in a catalytically active form, respectively, and wherein the plurality of cells further express a reporter gene in response to catalytic activity of the kinases;
wherein the plurality of cells are derived from a single cell type, and wherein cells expressing a first kinase of the plurality of kinases are separate from cells expressing a second kinase of the plurality of kinases;
acquiring with an acquisition system a first set of data elements from the cells expressing the first kinase and a second set of data elements from the cells expressing the second kinase, wherein first and second sets of data elements are measurements of signals generated by the reporter gene;
calculating a frequency of false positives and false negatives against a plurality of compounds to thereby validate first and second sets of data; and
calculating inhibition data from the first and second set of data elements.

28. The method of claim 27 wherein the first set of data elements of the first kinase is analyzed against the second set of data elements of the second kinase of the same or different kinase signaling pathway.

29. The method of claim 28 wherein the first and second kinase signaling pathways are selected from the group consisting of a MEK-ERK mediated pathway, a IKK-NFkB mediated pathway, a p38 mediated pathway, a JNK-Jun mediated pathway, and a JAK-STAT mediated pathway, and wherein the first and second kinase signaling pathways are same kinase signaling pathway.

30. The method of claim 28 wherein the first and second kinase signaling pathways are selected from the group consisting of a MEK-ERK mediated pathway, a IKK-NFkB mediated pathway, a p38 mediated pathway, a JNK-Jun mediated pathway, a PI3K-Akt mediated pathway, and a JAK-STAT mediated pathway, and wherein first and second kinase signaling pathways are not the same kinase signaling pathway.

31. The method of any one of claims 6, 13, or 20 wherein the first signal and the second signal comprises a radiologically detectable signal.
